(19) United States
(12) Patent Application Publication
(10) Pub. No.: US 2026/0128164 A1
(43) Pub. Date: May 7, 2026

(54) SYSTEMS AND METHODS FOR CLAIMS-BASED MEDICAL PROVIDER REASSIGNMENT

(71) Applicant: Aetna Inc., Hartford, CT (US)

(72) Inventors: John Wojciehowski, Hartford, CT (US); Naveen B. Rengan, Hartford, CT (US); Mrinal Joglekar, Hartford, CT (US); Rajesh Johnson, Hartford, CT (US)

(21) Appl. No.: 18/937,897

(22) Filed: Nov. 5, 2024

(51) Int. Cl. G16H 40/20; G16H 10/60

(52) U.S. Cl. CPC G16H 40/20; G16H 10/60

(57) ABSTRACT

In some instances, a method is provided. The method comprises: identifying a first member for reassignment based on determining that the first member is valid for reassignment; obtaining, from a claims repository, one or more claims for the first member within a first time period, wherein the one or more claims are medical claims associated with a primary care provider (PCP); based on the one or more claims indicating a second medical provider that is different from the first medical provider, reassigning the first member from the first medical provider to the second medical provider; and informing the first member of the reassignment from the first medical provider to the second medical provider.

### BACKGROUND

Members may visit medical providers for primary care that are different from the one assigned to them. For example, an enterprise organization may assign a medical provider to the member such as by using one or more automated processes. In some examples, the initial assignment may be inconvenient to the member for one or more reasons such as proximity to the member, continuity of care, and/or based on a family member of the member being assigned to a different medical provider. In such situations, the member may seek to change their assigned medical provider to another medical provider that is more convenient for them. However, the process may take time as many reassignment processes are conducted manually. For example, the member may be required to call a help desk, and then wait an amount of time before a new insurance card is issued indicating the reassignment of the medical provider. As such, in order to ensure that members received the best possible experience and health outcomes, there remains a technical need to automatically reassign medical providers to members.

### SUMMARY

In some examples, the present application uses an automated process for medical provider reassignment based on claims data for members. For example, members may be enrolled into an insurance plan provided by the enterprise organization. Additionally, and/or alternatively, a member may be enrolled into MEDICAID, and the enterprise organization may provide health insurance for the member. In a first step, the enterprise organization may assign the member a medical provider such as a primary care provider (PCP). However, due to one or more reasons and/or concerns, the member may seek to switch from their assigned medical provider to a different medical provider. For example, a family member (e.g., a spouse or a dependent of the member) may be assigned to a different medical provider, and the member may seek to be reassigned to the medical provider that the family member is assigned to. As such, the member may simply schedule a medical appointment with that medical provider, and after attending the medical appointment, the medical provider may provide claims data. Then, traditionally, the member may be forced to manually change their assigned medical provider so as to have their claims processed.

In contrast, the present application may include a claims-based reassignment computing platform that retrieves the claims data from a claims repository, and then automatically reassigns the member based on the claims data. For example, the claims-based reassignment computing platform may gather valid members for reassignment (e.g., if the member just became enrolled into MEDICAID, the claims-based reassignment computing platform may determine that the member is invalid for reassignment), and gather valid medical providers for reassignment. Then, the claims-based reassignment computing platform may gather valid claims for reassignment (e.g., from the claims repository) and perform a reassignment algorithm to reassign the valid members. For instance, in the example described above, the claims-based reassignment computing platform may gather the claims data associated with the member’s medical appointment, and then reassign the member to the new medical provider. This will be described in more detail below.

In one aspect, a method for reassigning a plurality of members from one or more currently assigned medical providers to one or more newly assigned medical providers is provided. The method comprises: identifying, by a claims-based reassignment computing platform, a first member for reassignment based on determining that the first member is valid for reassignment, wherein determining that the first member is valid for reassignment comprises: processing first member information to confirm that the first member is assigned to a first medical provider; determining that the first member has not been identified for reassignment by the claims-based reassignment computing platform within a first time window; and determining that an earliest effective enrollment date for the first member is outside of a second time window; obtaining, from a claims repository and by the claims-based reassignment computing platform, one or more claims for the first member within a first time period, wherein the one or more claims are medical claims associated with a primary care provider (PCP); based on the one or more claims indicating a second medical provider that is different from the first medical provider, reassigning, by the claims-based reassignment computing platform, the first member from the first medical provider to the second medical provider; and informing, by the claims-based reassignment computing platform, the first member of the reassignment from the first medical provider to the second medical provider.

Examples may include one of the following features, or any combination thereof. For example, in some instances, determining that the first member is valid for reassignment further comprises: determining that the first member has not been previously reassigned by the claims-based reassignment computing platform, and wherein determining that the earliest effective enrollment date for the first member is outside of the second time window is in response to determining that the first member has not been previously reassigned by the claims-based reassignment computing platform.

In some examples, the method further comprises: identifying, by the claims-based reassignment computing platform, a second member for reassignment based on determining that the second member is valid for reassignment, wherein determining that the second member is valid for reassignment comprises: processing second member information to confirm that the second member is assigned to a third medical provider; determining that the second member has not been identified for reassignment by the claims-based reassignment computing platform within the first time window; determining that the second member has been previously reassigned by the claims-based reassignment computing platform; and in response to determining that the second member has been previously reassigned, comparing a reassignment date for when the member was previously reassigned with a third time window; and based on determining that the second member is valid for reassignment, reassigning, by the claims-based reassignment computing platform, the second member from the third medical provider to a fourth medical provider associated with one or more second claims for the second member.

In some variations, the method further comprises: determining a third member is not valid for reassignment based on third member information indicating that the third member is enrolled into a first program and a second program.

In some instances, the method further comprises: determining a fourth member is not valid for reassignment based on fourth member information indicating that the fourth member is a newborn baby.

In some examples, the method further comprises: determining a fifth member is not valid for reassignment based on fifth member information indicating that the fifth member is currently assigned to a primary care provider (PCP) that has been terminated.

In some variations, processing the first member information to confirm that the first member is assigned to the first medical provider comprises processing the first member information to confirm that the first member is actively enrolled or has a future enrollment with a primary care provider (PCP).

In some instances, the method further comprises: determining a plurality of valid providers based on one or more provider criteria, and wherein obtaining the one or more claims for the first member is based on determining the plurality of valid providers.

In some examples, the one or more provider criteria indicates one or more panel thresholds, and wherein determining the plurality of valid providers comprises: comparing panel sizes indicating a number of members that are currently under care by the providers with the one or more panel thresholds; and determining the plurality of valid providers based on the comparison.

In some variations, obtaining the one or more claims for the first member within the first time period comprises: retrieving, from a claims systems, claims data comprising a plurality of claims associated with the first member; filtering the claims data to determine the one or more claims for the first member based on comparing dates of service for the plurality of claims with a fourth time window; and storing the one or more claims for the first member within a claims database, wherein reassigning the first member to the second medical provider is based on the one or more stored claims within the claims database.

In some instances, the one or more claims comprises a plurality of claims for the first member, and wherein reassigning the first member from the first medical provider to the second medical provider comprises: determining a claim, from the plurality of claims, that has a date of service that is most recent to a current run-time date, wherein the determined claim indicates the second medical provider; and reassigning the first member from the first medical provider to the second medical provider based on the determined claim indicating the second medical provider.

In some examples, the one or more claims comprises a plurality of claims for the first member, and wherein reassigning the first member from the first medical provider to the second medical provider comprises: determining a plurality of addresses associated with a plurality of providers that are indicated by the plurality of claims for the first member; based on comparing the plurality of addresses associated with the plurality of providers to an address associated with the first member, determining the second medical provider that has a closest geographical distance to the address associated with the first member; and reassigning the first member from the first medical provider to the second medical provider based on determining that the second medical provider the closest geographical distance to the address associated with the first member.

In some variations, the one or more claims comprises a plurality of claims for the first member, and wherein reassigning the first member from the first medical provider to the second medical provider comprises: determining a plurality of panel sizes associated with a plurality of providers that are indicated by the plurality of claims for the first member; based on a comparison of the plurality of panel sizes, determining the second medical provider that has a smallest panel size from the plurality of panel sizes; and reassigning the first member from the first medical provider to the second medical provider based on determining that the second medical provider has the smallest panel size from the plurality of panel sizes.

In some instances, informing the first member of the reassignment from the first medical provider to the second medical provider comprises performing a process to mail a new insurance card to the first member that indicates the second medical provider.

In another aspect, a computing platform for reassigning a plurality of members from one or more currently assigned medical providers to one or more newly assigned medical providers is provided. The computing platform comprises: one or more processors; and a non-transitory computer-readable medium having processor-executable instructions stored thereon, wherein the processor-executable instructions, when executed by the one or more processors, facilitate: identifying a first member for reassignment based on determining that the first member is valid for reassignment, wherein determining that the first member is valid for reassignment comprises: processing first member information to confirm that the first member is assigned to a first medical provider; determining that the first member has not been identified for reassignment by the computing platform within a first time window; and determining that an earliest effective enrollment date for the first member is outside of a second time window; obtaining, from a claims repository, one or more claims for the first member within a first time period, wherein the one or more claims are medical claims associated with a primary care provider (PCP); based on the one or more claims indicating a second medical provider that is different from the first medical provider, reassigning the first member from the first medical provider to the second medical provider; and informing the first member of the reassignment from the first medical provider to the second medical provider.

Examples may include one of the following features, or any combination thereof. For example, in some instances, determining that the first member is valid for reassignment further comprises: determining that the first member has not been previously reassigned by the computing platform, and wherein determining that the earliest effective enrollment date for the first member is outside of the second time window is in response to determining that the first member has not been previously reassigned by the computing platform.

In some examples, the processor-executable instructions, when executed by the one or more processors, further facilitate: identifying a second member for reassignment based on determining that the second member is valid for reassignment, wherein determining that the second member is valid for reassignment comprises: processing second member information to confirm that the second member is assigned to a third medical provider; determining that the second member has not been identified for reassignment by the computing platform within the first time window; determining that the second member has been previously reassigned by the computing platform; and in response to determining that the second member has been previously reassigned, comparing a reassignment date for when the member was previously reassigned with a third time window; and based on determining that the second member is valid for reassignment, reassigning the second member from the third medical provider to a fourth medical provider associated with one or more second claims for the second member.

In some variations, the processor-executable instructions, when executed by the one or more processors, further facilitate: determining a third member is not valid for reassignment based on third member information indicating that the third member is enrolled into a first program and a second program.

In some instances, the processor-executable instructions, when executed by the one or more processors, further facilitate: determining a fourth member is not valid for reassignment based on fourth member information indicating that the fourth member is a newborn baby.

In yet another aspect, a non-transitory computer-readable medium having processor-executable instructions stored thereon is provided. The processor-executable instructions, when executed, facilitate: identifying a first member for reassignment based on determining that the first member is valid for reassignment, wherein determining that the first member is valid for reassignment comprises: processing first member information to confirm that the first member is assigned to a first medical provider; determining that the first member has not been identified for reassignment by the computing platform within a first time window; and determining that an earliest effective enrollment date for the first member is outside of a second time window; obtaining, from a claims repository, one or more claims for the first member within a first time period, wherein the one or more claims are medical claims associated with a primary care provider (PCP); based on the one or more claims indicating a second medical provider that is different from the first medical provider, reassigning the first member from the first medical provider to the second medical provider; and informing the first member of the reassignment from the first medical provider to the second medical provider.

All examples and features mentioned above may be combined in any technically possible way.

### BRIEF DESCRIPTION OF THE DRAWINGS

The subject technology will be described in even greater detail below based on the exemplary figures, but is not limited to the examples. All features described and/or illustrated herein can be used alone or combined in different combinations. The features and advantages of various examples will become apparent by reading the following detailed description with reference to the attached drawings which illustrate the following:

FIG. 1 is a simplified block diagram depicting an exemplary computing environment in accordance with one or more examples of the present application.

FIG. 2 is a simplified block diagram of one or more devices or systems within the exemplary environment of FIG. 1.

FIG. 3 is an exemplary process for claims-based reassignment in accordance with one or more examples of the present application.

FIGS. 4A and 4B show exemplary flowcharts for claims-based reassignment in accordance with one or more examples of the present application.

### DETAILED DESCRIPTION

Examples of the presented application will now be described more fully hereinafter with reference to the accompanying FIGs., in which some, but not all, examples of the application are shown. Indeed, the application may be exemplified in different forms and should not be construed as limited to the examples set forth herein; rather, these examples are provided so that the application will satisfy applicable legal requirements. Where possible, any terms expressed in the singular form herein are meant to also include the plural form and vice versa, unless explicitly stated otherwise. Also, as used herein, the term “a” and/or “an” shall mean “one or more” even though the phrase “one or more” is also used herein. Furthermore, when it is said herein that something is “based on” something else, it may be based on one or more other things as well. In other words, unless expressly indicated otherwise, as used herein “based on” means “based at least in part on” or “based at least partially on”.

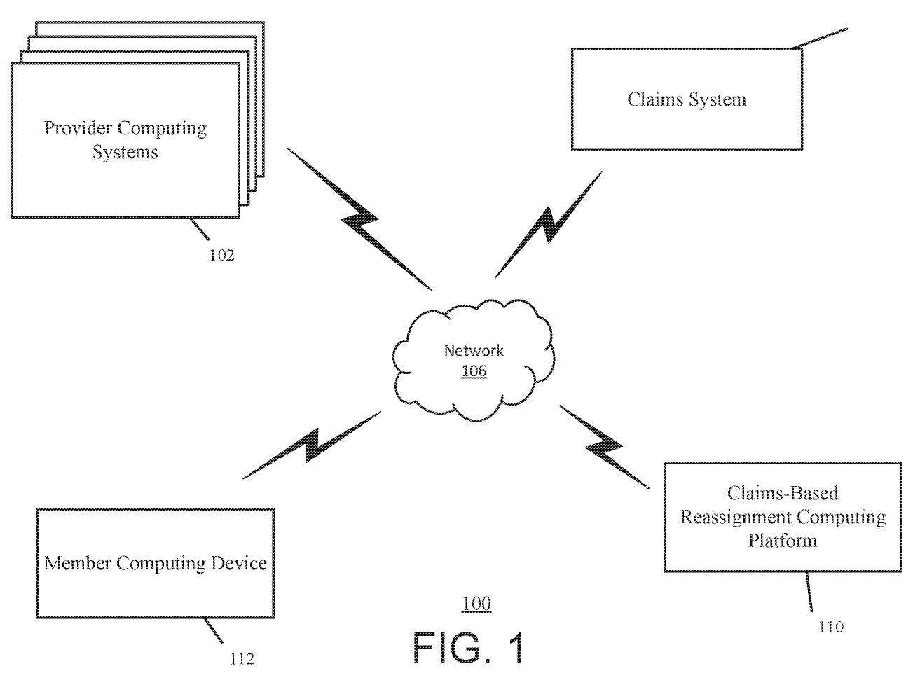

Systems, methods, and computer program products are herein disclosed that perform reassignment of medical providers based on claims data. FIG. 1 is a simplified block diagram depicting an exemplary environment in accordance with an example of the present application. The environment 100 includes provider computing systems 102, a network 106, a claims system 108, a claims-based reassignment computing platform 110, and a member computing device 112. The provider computing systems 102 may be associated with one or more providers. Although the entities within environment 100 may be described below and/or depicted in the FIGs. as being singular entities, it will be appreciated that the entities and functionalities discussed herein may be implemented by and/or include one or more entities.

The entities within the environment 100 such as the provider computing systems 102, the claims system 108, the claims-based reassignment computing platform 110, and/or the member computing device 112 may be in communication with other systems or facilities within the environment 100 via the network 106. The network 106 may be a global area network (GAN) such as the Internet, a wide area network (WAN), a local area network (LAN), or any other type of network or combination of networks. The network 106 may provide a wireline, wireless, or a combination of wireline and wireless communication between the entities within the environment 100. In some instances, one or more entities within the environment 100 may communicate with each other without using the network 106 (e.g., via communication protocols such as WI-FI or BLUTOOTH or via wired connections). For example, in some variations, the claims-based reassignment computing platform 110 may include the claims system 108. In other words, in such variations, the claims system 108 and the claims-based reassignment computing platform 110 might not be separate entities.

The provider computing systems 102 may be associated with one or more providers that (e.g., a variety of clinical providers, institutions, and/or facilities such as medical providers, treatment providers, and/or other types of clinical providers) may provide services to individuals (e.g., medical and behavioral health to individuals). For instance, a provider may be, include, and/or is otherwise associated with a physician (e.g., a primary care physician (PCP)), a group (e.g., an entity associated with a unique tax identification number (TIN) that accepts payments for services provided by a physician to a member at a location affiliated to the group), and/or a practice / service location (e.g., a physical office / location where a physician provides services to a member). For instance, in some examples, the provider may be a PCP such as a physician in internal medicine, family practice, general practice, pediatrics, obstetrician gynecologist (OB/GYN), geriatrics, and/or in a specialty such as cardiology, gastro enterology, and/or neurology. In some instances, the provider may satisfy one or more factors such as the provider is a participating provider (e.g., a participating physician (PAR), which may refer to a healthcare provider that has entered into an agreement with an insurance company or a healthcare payer to accept their predetermined payment rates for medical services rendered to patients covered by that particular insurance plan). Additionally, and/or alternatively, the factors may further include that the provider records with active contracts, the provider records with a PCP flag, the physician is affiliated with a group (e.g., a group having a particular TIN), the physician is affiliated with a designated practice/service location for a group, the practice/service location is affiliated with a group, the provider is not on “watch”, the physician is with an open panel (e.g., can “accept” new patients), and/or the physician is associated with a valid specialty.

For example, the individual may contact a provider and set-up an appointment with the provider. The provider may provide services (e.g., medical or behavioral services) to the individual such as providing treatment or diagnosis for a medical or behavioral condition. Further, the provider may generate information associated with the appointment such as generating claims data comprising claims (e.g., medical claims) for the appointment. The claims may be used to seek reimbursement from an enterprise organization such as an insurance enterprise organization. For example, the individual may be a member of a policy (e.g., insurance policy) that is provided by the enterprise organization. Based on reviewing the claims data, the enterprise organization may provide reimbursement for the service provided by the provider.

In some examples, the member may be assigned an initial provider. For example, the member may be covered by a policy of the enterprise organization based on a program such as MEDICAID. The enterprise organization may assign the member to the initial provider using one or more processes, systems, and/or methods (e.g., automated processes). For instance, a computing platform associated with the enterprise organization (e.g., the claims-based reassignment computing platform 110 and/or another computing platform) may perform an automated process to determine the initial provider for the member, and assign the member to the initial provider. However, in some instances and as mentioned above, the member may seek to use a different provider than the provider that was initially assigned by the enterprise organization. For example, the initial provider may be a substantial distance away from the residence of the member, and the member may seek a provider that is nearby to their domicile. Additionally, and/or alternatively, the member may be enrolled in MEDICAID, and may become eligible / ineligible during different durations. For instance, the member may be initially enrolled into MEDICAID and be assigned a first provider, but due to circumstances, may be ineligible for MEDICAID. Then, due to additional changing circumstances at a later time, the member may become eligible for MEDICAID again. When the member becomes eligible for MEDICAID again, a new provider may be assigned to the member, and to maintain continuity of care, the member may seek reassignment to the first provider. Additionally, and/or alternatively, the member may have a family member that is assigned to a different provider and may seek to switch from the initial provider that is assigned by the enterprise organization to the family member’s provider.

Thus, the member may set-up an appointment and attend the appointment with a provider that is different from their assigned provider (e.g., the initial provider). The provider may use a provider computing system 102 to provide claims data associated with the appointment, and the claims data may be stored in a database, such as the claims system 108. For example, the provider may include a provider computing system 102. The provider computing system 102 may be in communication with other entities from environment 100 such as the claims system 108. For instance, a clinician and/or another person associated with the provider may use the provider computing system 102 to generate information (e.g., claims data) associated with the member’s appointment. Afterwards, the provider computing system 102 may provide the generated information such as the claims data to another entity from environment 100 such as to the claims system 108.

The provider computing system 102 may be and/or include, but is not limited to, a desktop, laptop, tablet, mobile device (e.g., smartphone device, or other mobile device), smart watch, an internet of things (IOT) device, or any other type of computing device that generally comprises one or more communication components, one or more processing components, and one or more memory components. The provider computing system 102 may be able to execute software applications managed by, in communication with, and/or otherwise associated with the enterprise organization. Additionally, and/or alternatively, the provider computing system 102 may be configured to perform other functions.

The claims system 108 may obtain claims data from the provider computing systems 102, and store the claims data. The claims system 108 may include one or more devices, computing platforms, repositories, databases, systems, and/or other entities that store claims data from the provider computing systems 102. In some examples, the claims system 108 might not be separate from the claims-based reassignment computing platform 110. For instance, in such examples, the claims-based reassignment computing platform 110 may include the claims system 108 (e.g., a repository that includes the claims data from the provider computing systems 102).

The claims-based reassignment computing platform 110 is a computing platform that is associated with the enterprise organization. The enterprise organization may be any type of corporation, company, organization, and/or other institution. In some instances, the enterprise organization may own, operate, and/or be otherwise associated with one or more retail facilities (e.g., storefronts) and/or distribution centers. For instance, the enterprise organization may operate physical storefronts that sell a plurality of products (e.g., toothbrush, toothpaste, and so on). Additionally, and/or alternatively, the enterprise organization may provide policies such as insurance policies to members. The enterprise organization may review claims data and provide reimbursements for appointments with providers such as medical and/or behavioral reimbursements.

Further, as mentioned above, the enterprise organization (e.g., using the claims-based reassignment computing platform 110 or another computing platform) may assign an initial provider to the member. Then, due to one or more reasons, the member may use a different provider from the initial provider assigned by the enterprise organization, and the different provider may provide claims data to the claims system 108. Using the claims data, the claims-based reassignment computing platform 110 may reassign the member automatically to this new provider using one or more processes such as processes 300 and/or 400 described below. Additionally, and/or alternatively, the claims-based reassignment computing platform 110 may provide an indication to a member computing device 112 indicating the reassignment.

In some examples, the claims-based reassignment computing platform 110 may include a computing system that is used for providing members with new insurance cards. For example, the claims-based reassignment computing platform 110 may further automate the generation of a new insurance card for the member, and may mail the new insurance card to the member. As such, based on the member attending an appointment with a new provider that is not their assigned provider and based on performing processes 300 and/or 400 described below, the claims-based reassignment computing platform 110 may automatically reassign the member to the new provider and may further mail the member a new insurance card indicating the new provider.

The claims-based reassignment computing platform 110 includes one or more computing devices, computing systems, cloud computing platforms, systems, servers, and/or other apparatuses capable of performing tasks, functions, and/or other actions for the enterprise organization. In some variations, the claims-based reassignment computing platform 110 and/or aspects of the claims-based reassignment computing platform 110 may be implemented as engines, software functions, and/or applications. In other words, functionalities of the claims-based reassignment computing platform 110 may be implemented as software instructions stored in storage (e.g., memory) and executed by one or more processors.

The member computing device 112 may be a computing device that is associated with a member. For instance, as mentioned above, the member may attend an appointment with a new provider and the claims-based reassignment computing platform 110 may reassign the member to the new provider. The claims-based reassignment computing platform 110 may provide an indication to the member computing device 112 of the reassignment.

The member computing device 112 is and/or includes, but is not limited to, a desktop, laptop, tablet, mobile device (e.g., smartphone device, or other mobile device), smart watch, an internet of things (IOT) device, or any other type of computing device that generally comprises one or more communication components, one or more processing components, and one or more memory components. The member computing device 112 may be able to execute software applications and/or programs associated with the enterprise organization. Additionally, and/or alternatively, the member computing device 112 may be configured to operate a web browser to connect to a web page and/or applications hosted and/or managed by systems.

It will be appreciated that the exemplary environment depicted in FIG. 1 is merely an example, and that the principles discussed herein may also be applicable to other situations—for example, including other types of institutions, organizations, devices, systems, and network configurations.

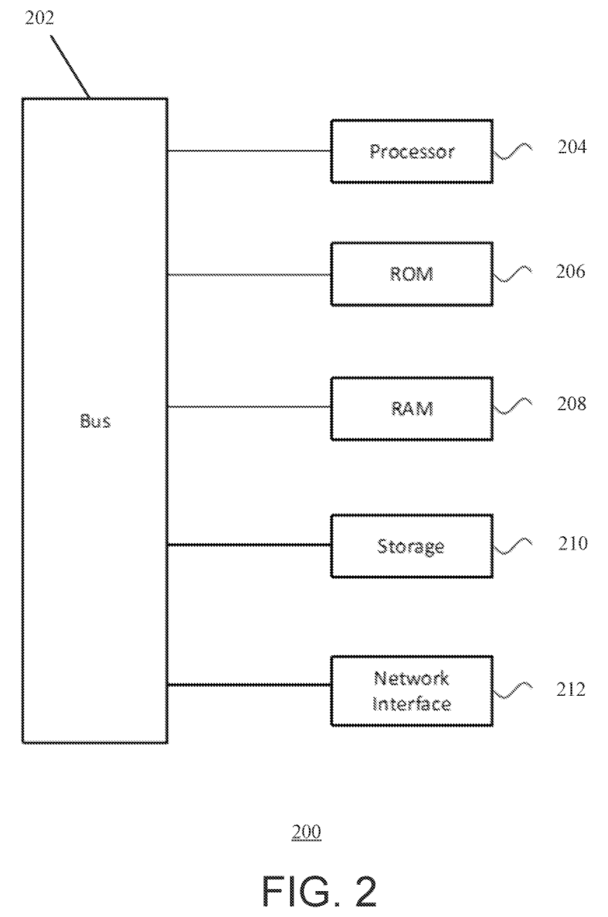

FIG. 2 is a block diagram of an exemplary system and/or device 200 within the environment 100. The device / system 200 includes a processor 204, such as a central processing unit (CPU), controller, and/or logic, that executes computer executable instructions for performing the functions, processes, and/or methods described herein. In some examples, the computer executable instructions are locally stored and accessed from a non-transitory computer readable medium, such as storage 210, which may be a hard drive or flash drive. Read Only Memory (ROM) 206 includes computer executable instructions for initializing the processor 204, while the random-access memory (RAM) 208 is the main memory for loading and processing instructions executed by the processor 204. The network interface 212 may connect to a wired network or cellular network and to a local area network or wide area network, such as the network 106. The device / system 200 may also include a bus 202 that connects the processor 204, ROM 206, RAM 208, storage 210, and/or the network interface 212. The components within the device / system 200 may use the bus 202 to communicate with each other. The components within the device / system 200 are merely exemplary and might not be inclusive of every component within the device / system 200.

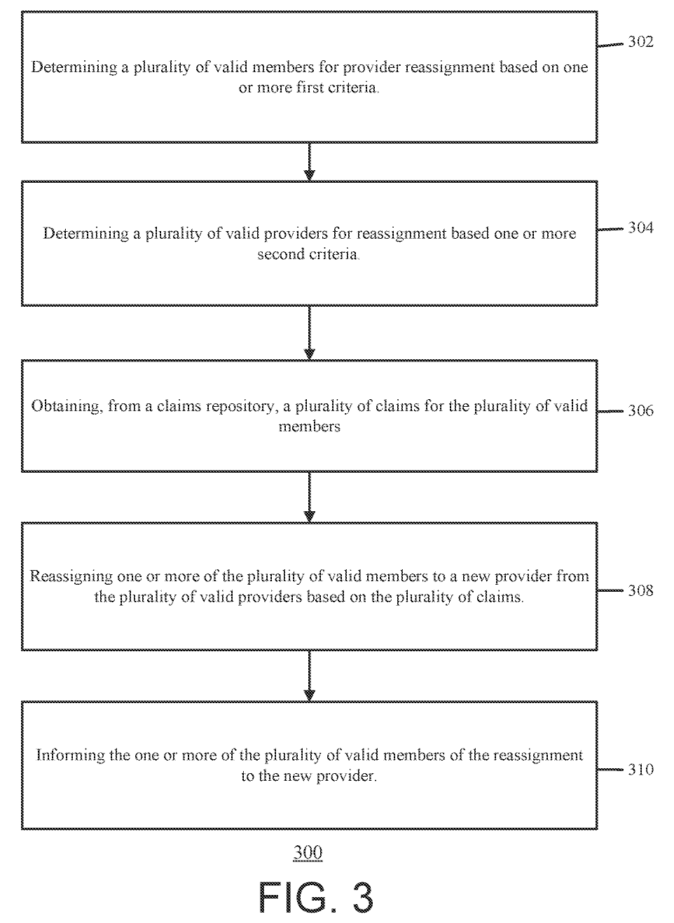

FIG. 3 is an exemplary process for claims-based reassignment in accordance with one or more examples of the present application. The process 300 may be performed by the claims-based reassignment computing platform 110 shown in FIG. 1. However, it will be recognized that any of the following blocks may be performed in any suitable order, the blocks may be performed by any suitable system, and that the process 300 may be performed in any suitable environment. The descriptions, illustrations, and processes of FIG. 3 are merely exemplary and the process 300 may use other descriptions, illustrations, and processes.

For example, as mentioned previously, members may be assigned to an initial provider (e.g., a first provider), but may attend an appointment with a different provider (e.g., a second provider) due to one or more reasons. The new provider may generate claims data and provide the claims data to the claims system 108. Based on the claims from the claims data associated with the member, process 300 may be performed to reassign the member to a new provider. In some examples, process 300 may be performed periodically (e.g., every day at a set time).

At block 302, the claims-based reassignment computing platform 110 determines a plurality of valid members for provider reassignment based on one or more first criteria. For example, the claims-based reassignment computing platform 302 may gather (e.g., determine, identify, retrieve, and/or obtain) valid member enrollments from a list of member enrollments for the enterprise organization (e.g., a list of total member enrollments for the enterprise organization or a list of a subset of member enrollments for the enterprise organization such as a list of all MEDICAID member enrollments). For instance, the claims-based reassignment computing platform 302 may retrieve a list of member enrollments for the enterprise organization and filter the list of member enrollments using the one or more first criteria. The claims-based reassignment computing platform 302 may determine the valid members for provider reassignment based on the filtering (e.g., the members from the list meeting the one or more first criteria).

In some examples, the one or more first criteria may indicate whether the member was previously gathered using process 300 and there were no current / qualifying claims associated with the member, a time duration that the member has been enrolled into the policy being provided by the enterprise organization, and/or whether the member was previously reassigned using process 300 within a certain time window. For example, based on the member being enrolled into the policy for a certain time period (e.g., ninety days), the claims-based reassignment computing platform 302 may determine the member as a valid member. Additionally, and/or alternatively, based on the member being previously reassigned using process 300 within a certain time window, the claims-based reassignment computing platform 302 may determine the member as not a valid member (e.g., excluding the member from being gathered again as a valid member for performing reassignment). Additionally, and/or alternatively, based on the member being previously gathered by process 300, but having indicated that there were no current / qualifying claims from the claims system 108, the claims-based reassignment computing platform 302 may determine the member as not a valid member (e.g., excluding the member from being gathered again as a valid member for performing reassignment). For instance, if previously, the claims-based reassignment computing platform 302 performed process 300 and attempted to gather valid claims for the member, but failed to obtain any valid claims (e.g., the member had not gone to any medical appointments at that time), the claims-based reassignment computing platform 302 might not reassign the member to a new provider and may exclude the member from being gathered again for a set time period.

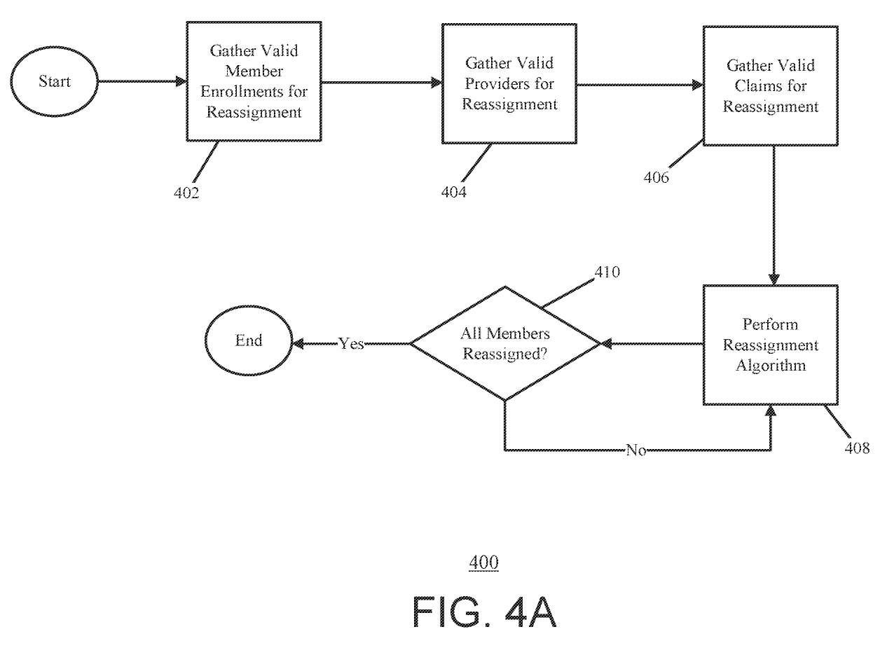

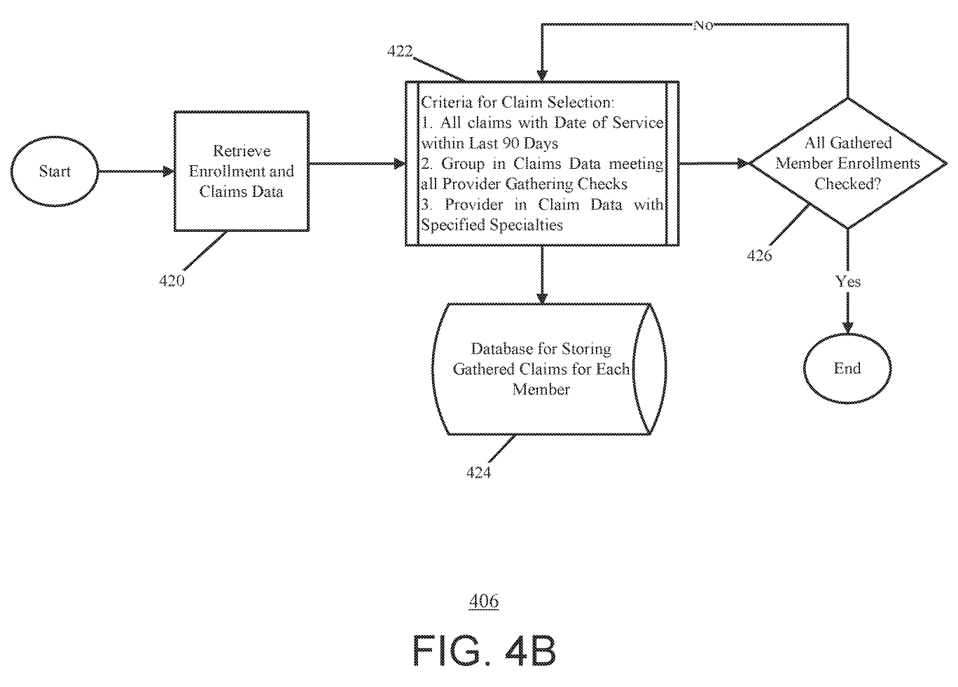

In other words, in some variations, the claims-based reassignment computing platform 110 may identify one or more members (e.g., a first member) for reassignment based on determining that the first member is valid for reassignment. The first member being valid for reassignment may include the claims-based reassignment computing platform 110 processing first member information (e.g., information associated with the member) to confirm that the first member is actively enrolled in a program associated with the enterprise organization (e.g., MEDICAID) and is assigned to a first medical provider (e.g., the initial medical provider). Furthermore, the claims-based reassignment computing platform 110 may determine that the first member has not been identified for reassignment by the claims-based reassignment computing platform 110 within a first time window and that an earliest effective enrollment date for the first member is outside of a second time window. Based on the determinations and/or confirmations, the claims-based reassignment computing platform 110 may determine the first member is valid for reassignment. Block 302 and the first criteria will be described in further detail below (e.g., block 402 of FIG. 4A).

At block 304, the claims-based reassignment computing platform 110 determines a plurality of valid providers for reassignment based on one or more second criteria. For example, the claims-based reassignment computing platform 110 may retrieve a list of providers that are associated with the enterprise organization (e.g., known to the enterprise organization). Then, using one or more second criteria, the claims-based reassignment computing platform 110 may filter the list of providers to a list of valid providers. For example, each provider may have corresponding information describing the provider such as whether they are “active.” The claims-based reassignment computing platform 110 may filter the providers based on the corresponding information associated with the providers. This will be described in further detail below (e.g., block 404 of FIG. 4A).

At block 306, the claims-based reassignment computing platform 110 obtains, from a claims repository (e.g., the claims system 108), a plurality of claims for the plurality of valid members. For example, the claims system 108 may include claims data for a plurality of members. The claims-based reassignment computing platform 110 may retrieve claims from the claims data, and determine the claims from the plurality of valid members that was determined in block 302. For example, each of the claims within the claims data may indicate a date of service and an identification of a member that is being serviced. Based on the date of service and/or the identification of the member, the claims-based reassignment computing platform 110 may determine to retrieve the claim. Additionally, and/or alternatively, the claims-based reassignment computing platform 110 may retrieve all claims from the claims system 108 based on the valid members, and then filter the retrieved claims based on the date of service of the claims, and/or vice versa. In some examples, the claims-based reassignment computing platform 110 may use additional and/or alternative factors to determine and/or obtain the valid claims from the claims repository. Block 306 will be described in further detail below (e.g., block 406 of FIG. 4A).

At block 308, the claims-based reassignment computing platform 110 reassigns one or more of the plurality of valid members to a new provider from the plurality of valid providers based on the plurality of claims. For example, based on a claim retrieved from block 306, the claims-based reassignment computing platform 110 may determine a new provider for the member that is not their previously assigned provider. The claims-based reassignment computing platform 110 may reassign the new provider to the member as their assigned provider. In some examples, the claims-based reassignment computing platform 110 may obtain multiple claims indicating multiple different providers that the member went to. The claims-based reassignment computing platform 110 may select a provider from the multiple different providers based on one or more third criteria. For instance, the claims-based reassignment computing platform 110 may select the provider associated with the claim with the most recent date of service. Additionally, and/or alternatively, the claims-based reassignment computing platform 110 may select the provider randomly from the multiple different providers. In some variations, the claims-based reassignment computing platform 110 may perform a check on the provider prior to the reassignment. For instance, based on an age, gender, and/or panel size restriction, the claims-based reassignment computing platform 110 might not reassign the member to the identified provider. Block 308 will be described in further detail below (e.g., block 408 of FIG. 4A).

At block 310, the claims-based reassignment computing platform 110 informs the one or more of the plurality of valid members of the reassignment to the new provider. For instance, the claims-based reassignment computing platform 110 may provide an indication to a member computing device 112 indicating the reassignment to the new provider. For example, the claims-based reassignment computing platform 110 may provide a notification to the member computing device 112, and the notification may indicate the reassignment. Additionally, and/or alternatively, as mentioned above, the claims-based reassignment computing platform 110 may perform a process to mail a new insurance card to the member.

FIG. 4A is an exemplary flowchart 400 for claims-based reassignment in accordance with one or more examples of the present application. The flowchart 400 may be performed by the claims-based reassignment computing platform 110 shown in FIG. 1. However, it will be recognized that any of the following blocks may be performed in any suitable order, the blocks may be performed by any suitable system, and that the flowchart 400 may be performed in any suitable environment. The descriptions, illustrations, and processes of FIG. 4A are merely exemplary and the flowchart 400 may use other descriptions, illustrations, and processes.

At block 402, the claims-based reassignment computing platform 110 gathers valid member enrollments for reassignment. For instance, the claims-based reassignment computing platform 110 may gather members (e.g., new members and/or re-enrolling members) who have already been assigned to a provider (e.g., an assigned PCP). For example, in some variations, the claims-based reassignment computing platform 110 may be used to reassign members that are enrolled into MEDICAID. At block 402, the claims-based reassignment computing platform 110 may gather members that are new to MEDICAID and/or members that have been previously enrolled into MEDICAID and are reenrolling into MEDICAID. For instance, due to circumstances, members may become ineligible for MEDICAID, but at a later time, may become eligible for MEDICAID again. As such, the claims-based reassignment computing platform 110 may gather members that are new and/or reenrolled into MEDICCAID.

As mentioned above, the claims-based reassignment computing platform 110 may use one or more first criteria to determine whether to gather members for reassignment. In some examples, the one or more first criteria may include whether the members have an active and/or future enrollment with a provider on a run-date (e.g., run-date of process 300 and/or 400) and/or whether the claims-based reassignment computing platform 110 has previously gathered the member within a first time window. For example, the claims-based reassignment computing platform 110 may check whether a member such as the first member has an active and/or future enrollment with a PCP (e.g., the first member has a PCP currently assigned to them). In other words, if the first member does not have an active and/or future enrollment with a PCP, then the first member may not qualify for reassignment. As such, the claims-based reassignment computing platform 110 might not perform reassignment of providers for the first member. In some instances, the claims-based reassignment computing platform 110 may perform reassignment based on the member having an active enrollment as of the run date. For instance, based on the run date of July 22^{nd}, the claims-based reassignment computing platform 110 may determine that the member has an active enrollment if the member has an enrollment that started on July 22^{nd} and/or prior to July 22^{nd} but does not end until after July 22^{nd}. In some examples, the claims-based reassignment computing platform 110 may perform reassignment based on the member having a future enrollment as of the run date. For instance, based on a run date of July 22^{nd}, the claims-based reassignment computing platform 110 may determine that the member has a future enrollment based on having an enrollment that starts on July 23^{rd} or later and does not end until after July 23^{rd}. In some variations, the claims-based reassignment computing platform 110 may perform reassignment based on the member having an active and non-overlapping future enrollment as of the run date. For instance, based on a run date of July 22^{nd}, the claims-based reassignment computing platform 110 may determine that the member has an active and non-overlapping future enrollment based on having two enrollments without a gap (e.g., active enrollment from January 1^{st} to July 31^{st} and from August 1^{st} to a later date in the future) and/or having two enrollments with a gap (e.g., from January 1^{st} to July 31^{st} and from September 1^{st} to a later date in the future). In other words, the claims-based reassignment computing platform 110 may identify members that have an active enrollment or a future enrollment in a program associated with the enterprise organization (e.g., MEDICAID), and then gather the identified members based on this criteria and/or other criteria being satisfied.

Additionally, and/or alternatively, the claims-based reassignment computing platform 110 may check whether the member has been previously gathered using process 300 and/or 400. For example, the claims-based reassignment computing platform 110 may execute process 300 and/or 400 each day, and may have gathered the first member twenty days ago. But, due to one or more factors such as the claims-based reassignment computing platform 110 not being able to retrieve any claims for the first member (e.g., blocks 406 and 408 below), the claims-based reassignment computing platform 110 might not have re-assigned the member to a new provider. However, given that the member was gathered previously, the claims-based reassignment computing platform 110 may compare the instance (e.g., date) that the member was gathered (e.g., identified) with a first time window (e.g., thirty days), and based on the comparison, may determine whether to gather the member again at block 302. In other words, based on the claims-based reassignment computing platform 110 gathering the member twenty days ago, which is less than the first time window of thirty days, at block 302, the claims-based reassignment computing platform 110 might not gather the member in this iteration. Instead, after thirty days elapsed, the member may then become eligible to be gathered again.

Additionally, and/or alternatively, the one or more first criteria may further indicate whether the claims-based reassignment computing platform 110 has previously reassigned the member to a new provider. For example, the claims-based reassignment computing platform 110 may determine that the member (e.g., the first member) has not been previously reassigned to a new provider (e.g., the claims-based reassignment computing platform 110 may determine that the process 300 and/or 400 has not previously reassigned the first member to a new provider). Based on the determination that the member has not been previously reassigned, the claims-based reassignment computing platform 110 may compare the earliest enrollment effective date of the member with a second time window, and determine whether to gather the member for reassignment based on the comparison. For example, the member may have enrolled into MEDICAID sixty days ago. The claims-based reassignment computing platform 110 may compare the earliest enrollment effective date (e.g., sixty days ago) with the second time window (e.g., ninety days). Based on the comparison, the claims-based reassignment computing platform 110 may determine whether to gather the member. For example, based on the member not being enrolled for the entire duration of the second time window (e.g., ninety days), the claims-based reassignment computing platform 110 may determine to not gather the member for reassignment. Based on the member being enrolled for the second time window, the claims-based reassignment computing platform 110 may gather the member for reassignment. In other words, the claims-based reassignment computing platform 110 might not reassign a member if the member has recently enrolled within a program (e.g., MEDICAID) associated with the enterprise organization (e.g., within the last ninety days).

Based on the determination that the member has been previously reassigned, the claims-based reassignment computing platform 110 may compare the reassignment date of the member with a third time window, and determine whether to gather the member for reassignment based on the comparison. For example, the claims-based reassignment computing platform 110 may have reassigned the member twenty-five days ago and the third time window may be thirty days. Based on the comparison, the claims-based reassignment computing platform 110 may determine that the claims-based reassignment computing platform 110 has previously reassigned the member within the third time window, and might not gather the member for reassignment. However, based on the claims-based reassignment computing platform 110 determining that the member was previously reassigned outside of the third time window (e.g., forty days ago), the claims-based reassignment computing platform 110 may gather the member for reassignment again. In other words, the claims-based reassignment computing platform 110 might not gather a member if the member has been recently reassigned to a new provider by the claims-based reassignment computing platform 110 using process 300 and/or 400.

Additionally, and/or alternatively, the one or more first criteria may include whether the member is enrolled in one or more programs associated with the enterprise organization. For example, the claims-based reassignment computing platform 110 may determine whether the member is enrolled into a first program (e.g., MEDICAID) and whether the member is enrolled into a second program (e.g., MEDICARE). Based on being enrolled in two programs, the claims-based reassignment computing platform 110 may determine whether to gather the member. For example, based on being enrolled in both MEDICAID and MEDICARE, the claims-based reassignment computing platform 110 might not gather the member for reassignment. In other words, the claims-based reassignment computing platform 110 may determine that given the member is enrolled into MEDICARE, the claims-based reassignment computing platform 110 might not gather the member for reassignment.

Additionally, and/or alternatively, the one or more first criteria may include whether the member is a recently born baby. For example, based on an identifier associated with the member (e.g., an identifier indicating that the member is a baby that was recently born), the claims-based reassignment computing platform 110 might not gather the member for reassignment.

Additionally, and/or alternatively, the one or more first criteria may include whether the active enrollment for the member has a terminated provider (e.g., terminated PCP). For instance, based on the provider being terminated (e.g., the PCP is no longer a valid provider for MEDICAID), the claims-based reassignment computing platform 110 might not gather the member for reassignment. In other words, based on a flag indicating that the provider that is currently assigned to the member has been terminated or voided, the claims-based reassignment computing platform 110 might not gather the member for reassignment.

Additionally, and/or alternatively, the one or more first criteria may include whether one or more flags have been provided about the member. For instance, based on previous interactions, the claims-based reassignment computing platform 110 and/or another computing platform associated with the enterprise organization may set a flag for the member. The claims-based reassignment computing platform 110 might not gather the member for reassignment based on the flag.

At block 404, the claims-based reassignment computing platform 110 gathers valid providers (e.g., from the providers) for reassignment. For example, the claims-based reassignment computing platform 110 may gather providers where their PCP, Practice Location, and Group are marked as “active” or “PCP.” For example, not every provider may qualify for the reassignment process 300 and/or 400 and the providers may have to meet one or more checks (e.g., second criteria). For instance, as mentioned above, the provider may be associated with a practitioner, group, and/or service location (e.g., facility). The claims-based reassignment computing platform 110 may gather a provider such as based on determining relationships between the practitioner, group, and/or service location. In some instances, a provider flag may be set for the provider (e.g., a practitioner or a service location) such as the practitioner having a “watch (e.g., a provider with a situation). The claims-based reassignment computing platform 110 may check the provider flag as well as the relationships associated with the provider associated with the provider flag (e.g., a practitioner may have a provider flag, and the claims-based reassignment computing platform 110 may check the group and/or service location associated with the practitioner). Based on the check, the claims-based reassignment computing platform 110 may determine whether the provider is valid for use in reassignment.

Additionally, and/or alternatively, each provider may be associated with a panel (e.g., panel size), and the claims-based reassignment computing platform 110 may gather the valid providers based on the panel. For example, each provider (e.g., practitioner, group, and/or service location) may be associated with a panel (e.g., a number of members that are currently under care of the provider). For instance, the claims-based reassignment computing platform 110 may compare the panels associated with the providers with one or more panel thresholds (e.g., 100), and may gather the valid providers based on the comparison. In other words, the claims-based reassignment computing platform 110 might not seek to gather providers that are at capacity or are close to capacity (e.g., providing care to 100 or more members already). Therefore, the claims-based reassignment computing platform 110 may compare the panel of the provider with the panel threshold to determine whether the provider is at capacity or are close to capacity. The claims-based reassignment computing platform 110 may gather valid providers that are not at capacity or are not close to capacity.

At block 406, the claims-based reassignment computing platform 110 gathers valid claims for reassignment. For instance, the claims-based reassignment computing platform 110 may gather (e.g., retrieve) claims from the claims system 108. For example, the claims-based reassignment computing platform 110 may retrieve claims from the claims system 108 based on one or more third criteria such as a date of service of the claims. For instance, the claims data may indicate a date of service (e.g., when the member attended the appointment with the provider). The claims-based reassignment computing platform 110 may retrieve claims based on the date of service being within a fourth time window (e.g., a time period such as within the last ninety days). For example, based on the determined valid members and/or valid providers from blocks 402 and 404, the claims-based reassignment computing platform 110 may gather claims for the valid members and/or valid providers that are within the date of service.

Additionally, and/or alternatively, the claims-based reassignment computing platform 110 may gather claims data associated with providers in specified specialties. For example, the claims data may indicate a provider that provided the service, and the provider may be associated with a certain specialty (e.g., obstetrician / gynecologist (OBGYN)). The claims-based reassignment computing platform 110 may gather all claims associated with the specialty (e.g., all claims for providers indicating the specialty). For example, based on the determined valid members and/or valid providers from blocks 402 and 404, the claims-based reassignment computing platform 110 may gather claims for the valid members and/or valid providers that are for the specialty.

Additionally, and/or alternatively, the claims-based reassignment computing platform 110 may gather claims data that meets all provider gathering checks. For example, the claims-based reassignment computing platform 110 may gather a claim based on checking whether a provider (e.g., practitioner) is associated with a primary specialty code (e.g., indicating that the practitioner is associated with a specialty), a claim start date that is non-null, a group identified via the claims exists within a contract information database and is active as of the run-date, and/or other provider gathering checks. Block 406 will be described in further detail in FIG. 4B.

FIG. 4B describes an exemplary implementation of the claims-based reassignment computing platform 110 performing block 406 of process 400. However, it will be recognized that any of the following blocks from FIG. 4B may be performed in any suitable order. The descriptions, illustrations, and processes of FIG. 4B are merely exemplary and the claims-based reassignment computing platform 110 may perform block 406 using other descriptions, illustrations, and processes.

At block 420, the claims-based reassignment computing platform 110 may retrieve enrollment and claims data. For example, based on the valid members determined at block 302, the claims-based reassignment computing platform 110 may retrieve claims data associated with the valid members from the claims system 108.

At block 422, the claims-based reassignment computing platform 110 may filter the retrieved claims data based on one or more criteria. For instance, the criteria may include, but are not limited to, all claims with a date of service within last ninety days, group in claims data meeting all provider gathering checks, and/or the provider from the claims data is within a specified specialty. The criteria for block 422 (e.g., the one or more third criteria) is described above.

Based on the claims-based reassignment computing platform 110 determining that one or more claims from the claims data satisfies the criteria, the claims-based reassignment computing platform 110 may store the claims (e.g., qualified claims) in the database 424. For instance, the database 424 may be used to store gathered claims for each member. In some instances, the database 424 may be part of the claims-based reassignment computing platform 110. In other instances, the database 424 may be separate from the claims-based reassignment computing platform 110.

At block 426, the claims-based reassignment computing platform 110 may determine whether all gathered member enrollments have been checked. For instance, the claims-based reassignment computing platform 110 may check whether the valid claims for each valid member, which were gathered / determined at block 402, have been obtained and stored within the database 424. Based on determining that there are additional valid claims for valid members from block 402, the claims-based reassignment computing platform 110 may determine no, and move back to block 422. Based on determining that all valid claims for the valid members were gathered and stored in the database 424, the claims-based reassignment computing platform 110 may determine yes, and proceed to the end of block 406.

Returning back to FIG. 4A, at block 408, the claims-based reassignment computing platform 110 performs a reassignment algorithm to reassign the members based on the valid member enrollments, the valid providers, and the valid claims. For example, for each valid member, the claims-based reassignment computing platform 110 may determine the retrieved claims for the member. Then, the claims-based reassignment computing platform 110 may assign a new provider to the member based on the retrieved claims. For example, based on the member having a single valid claim (e.g., a single claim within the fourth time window such as ninety days), the claims-based reassignment computing platform 110 may check the claim to determine the provider (e.g., practitioner, group, and/or service location) associated with the claim. The claims-based reassignment computing platform 110 may reassign the member from the initial provider to the determined provider associated with the claim.

In some examples, based on having multiple “valid” providers gathered for a member within the retrieved claims, the claims-based reassignment computing platform 110 may determine whether the provider from the most recent claim (e.g., the claim that has a date of service closest to the current run time date) is a “valid” provider (e.g., within the list of valid providers determined from block 404). Based on the provider being a valid provider, the claims-based reassignment computing platform 110 sets the provider as the new provider for the member. In other words, the member may have visited several (e.g., five) different providers, which issued five claims within the third time window. The claims-based reassignment computing platform 110 may have gathered all five claims from the five different providers. Then, based on a date-of-service associated with the claims, the claims-based reassignment computing platform 110 may determine the provider associated with the closest date of service to the current run time date. For example, the member may have visited four of the five providers several months ago, but may have visited the fifth provider last week. As such, the claims-based reassignment computing platform 110 may determine the date of service that is closest to the current run time date as the fifth provider, and may set the fifth provider as the new provider for the member.

In some instances, based on the provider not being a valid provider, the claims-based reassignment computing platform 110 may maintain the same provider as previously assigned. For example, the claims-based reassignment computing platform 110 may determine that the only claim for the member or that the most recent claim for the member (e.g., the fifth provider in the example above) indicates a provider that is not a valid provider (e.g., based on comparing the provider indicated by the claim with the valid providers that were gathered at block 404). Based on the determination, the claims-based reassignment computing platform 110 may keep the initial provider (e.g., not assign the member to the new provided indicated by the claim). Additionally, and/or alternatively, even though the claims-based reassignment computing platform 110 might not reassign the member to a new provider, the claims-based reassignment computing platform 110 may set a flag or indication such that the member is not gathered again by process 300 and/or 400 for a set duration (e.g., thirty days). For example, as mentioned above, at block 402, the claims-based reassignment computing platform 110 may check whether the member has been previously gathered using process 300 and/or 400, but not reassigned. Thus, based on the provider associated with the claim not being a valid provider, the claims-based reassignment computing platform 110 may determine that the member was gathered, and not reassigned. The claims-based reassignment computing platform 110 may then wait the first time window (e.g., thirty days) prior to the member being eligible to be gathered again for reassignment.

In some variations, based on the member having multiple claims indicating the same date of service, and the claims are associated with different valid providers, the claims-based reassignment computing platform 110 may select a provider from the multiple different providers using one or more algorithms (e.g., randomly such as by using a random number generator). For example, in some instances, the claims-based reassignment computing platform 110 may perform a distance check and determine the provider that is closest in distance (e.g., geographical distance) to the address associated with the member. For instance, the claims-based reassignment computing platform 110 may determine the address of the member (e.g., home residence address of the member) and addresses associated with the providers, which may be indicated in the claims data / claims. Based on a comparison between the addresses, the claims-based reassignment computing platform 110 may determine the provider that is closest in distance to the address associated with the member, and reassign the member to the determined provider.

Additionally, and/or alternatively, the claims-based reassignment computing platform 110 may determine a smallest panel size associated with the providers and assign the member to the provider with the smallest panel size. For instance, as mentioned above, the panel size may be associated with a number of members that are currently under care by the provider. The claims-based reassignment computing platform 110 may determine the panel sizes for all of the providers, compare the panel sizes to each other, and determine a provider with the smallest panel size. The claims-based reassignment computing platform 110 may reassign the member to the determined provider.

In some examples, after determining the new provider for the reassignment, the claims-based reassignment computing platform 110 may check whether the new provider is part of the same group as the initial provider. For example, as mentioned above, the providers may be associated with a group that has the same tax number (e.g., same TIN). The claims-based reassignment computing platform 110 may check whether the new provider (e.g., practitioner or PCP) has the same tax number (e.g., same TIN) as the initial provider (e.g., the initial practitioner or PCP). For instance, the initial PCP and the newly determined PCP may be part of the same group (e.g., may have the same tax number). Based on the new provider being part of the same group as the initial provider, the claims-based reassignment computing platform 110 might not reassign the member to the new provider (e.g., keep the initial provider). Based on the new provider being part of a different group than the initial provider, the claims-based reassignment computing platform 110 may reassign the member to the new provider. In other words, if the new PCP that was determined from the claims information is part of the same tax group as the initial PCP, the claims-based reassignment computing platform 110 might not make a switch to the new provider / PCP. If the new PCP and initial PCP are part of different tax groups, the claims-based reassignment computing platform 110 may reassign the member to the new provider / PCP.

In some variations, prior to confirming the assignment for switching, the claims-based reassignment computing platform 110 may check for age and/or gender restrictions associated with the determined provider. For instance, the determined provider may have an age requirement (e.g., provider only accepts members within a certain age range) and/or gender restriction (e.g., the provider is not accepting males, females, and/or both genders). Based the provider’s age or gender restrictions, the claims-based reassignment computing platform 110 might not assign the member to the new provider. Additionally, and/or alternatively, the claims-based reassignment computing platform 110 may check for panel size associated with the determined provider. For instance, providers may indicate a maximum assignment limit (e.g., panel size), which allows for a little “room” for further manual assignment / file assignment to that provider based on member election. As such, the claims-based reassignment computing platform 110 may check for panel size associated with the selected provider, and based on the provider being at or exceeding the elected panel size, the claims-based reassignment computing platform 110 might not assign the member to the provider.

Based on confirming that the claims-based reassignment computing platform 110 may reassign the member to the determined provider, the claims-based reassignment computing platform 110 may terminate the current provider (e.g., the current PCP may be terminated to one day before the run date), delete the future providers (e.g., the future PCPs may be deleted), and add the new provider (e.g., the member may be reassigned to the new provider that is determined in blocks 406 and 408).

At block 410, the claims-based reassignment computing platform 110 checks whether all members from the gathered valid members are reassigned. If not, flowchart 400 moves back to block 408. If so, flowchart 400 ends. For instance, based on gathering the valid members, valid providers, and valid claims, the claims-based reassignment computing platform 110 performs block 408 to reassign the valid members with valid providers using the valid claims. At block 410, the claims-based reassignment computing platform 110 checks whether all valid members have been reassigned. If not, the claims-based reassignment computing platform 110 repeats block 408 until all valid members have been reassigned.

In some variations, flowchart 400 may be executed daily. Additionally, and/or alternatively, the claims-based reassignment computing platform 110 may perform additional functions such as performing auto-assigning of new / reenrolled members with providers. In some instances, the claims-based reassignment computing platform 110 may check whether any additional functions (e.g., performing auto-assigning of new / reenrolled members with providers) are currently being performed. If so, the claims-based reassignment computing platform 110 may wait to perform flowchart 400 until the other function has completed its performance.

In some examples, after performing flowchart 400, the claims-based reassignment computing platform 110 may generate a report, which indicates a list of members that were successfully reassigned to one or more providers. Additionally, and/or alternatively, the report may further indicate members that need to have their providers changed manually. Additionally, and/or alternatively, the report may further indicate failures due to a bad environment, due to server unavailability, and/or due to processing by an enrollment manager.

A number of implementations have been described. Nevertheless, it will be understood that additional modifications may be made without departing from the scope of the inventive concepts described herein, and, accordingly, other examples are within the scope of the following claims. For example, it will be appreciated that the examples of the application described herein are merely exemplary. Variations of these examples may become apparent to those of ordinary skill in the art upon reading the foregoing description. The inventor expects skilled artisans to employ such variations as appropriate, and the inventor intends for the application to be practiced otherwise than as specifically described herein. Accordingly, this application includes all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

It will further be appreciated by those of skill in the art that the execution of the various machine-implemented processes and steps described herein may occur via the computerized execution of processor-executable instructions stored on a non-transitory computer-readable medium, e.g., random access memory (RAM), read-only memory (ROM), programmable read-only memory (PROM), volatile, nonvolatile, or other electronic memory mechanism. Thus, for example, the operations described herein as being performed by computing devices and/or components thereof may be carried out by according to processor-executable instructions and/or installed applications corresponding to software, firmware, and/or computer hardware.

The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the application and does not pose a limitation on the scope of the application unless otherwise claimed. No language in the specification should be construed as indicating any non-claimed element as essential to the practice of the application.

1. A method for reassigning a plurality of members from one or more currently assigned medical providers to one or more newly assigned medical providers, comprising:
identifying, by a claims-based reassignment computing platform, a first member for reassignment based on determining that the first member is valid for reassignment, wherein determining that the first member is valid for reassignment comprises:
processing first member information to confirm that the first member is assigned to a first medical provider;
determining that the first member has not been identified for reassignment by the claims-based reassignment computing platform within a first time window; and
determining that an earliest effective enrollment date for the first member is outside of a second time window;
obtaining, from a claims repository and by the claims-based reassignment computing platform, one or more claims for the first member within a first time period, wherein the one or more claims are medical claims associated with a primary care provider (PCP);
based on the one or more claims indicating a second medical provider that is different from the first medical provider, reassigning, by the claims-based reassignment computing platform, the first member from the first medical provider to the second medical provider; and
informing, by the claims-based reassignment computing platform, the first member of the reassignment from the first medical provider to the second medical provider.

2. The method of claim 1, wherein determining that the first member is valid for reassignment further comprises:
determining that the first member has not been previously reassigned by the claims-based reassignment computing platform, and wherein determining that the earliest effective enrollment date for the first member is outside of the second time window is in response to determining that the first member has not been previously reassigned by the claims-based reassignment computing platform.

3. The method of claim 1, further comprising:
identifying, by the claims-based reassignment computing platform, a second member for reassignment based on determining that the second member is valid for reassignment, wherein determining that the second member is valid for reassignment comprises:
processing second member information to confirm that the second member is assigned to a third medical provider;
determining that the second member has not been identified for reassignment by the claims-based reassignment computing platform within the first time window;
determining that the second member has been previously reassigned by the claims-based reassignment computing platform; and
in response to determining that the second member has been previously reassigned, comparing a reassignment date for when the member was previously reassigned with a third time window; and
based on determining that the second member is valid for reassignment, reassigning, by the claims-based reassignment computing platform, the second member from the third medical provider to a fourth medical provider associated with one or more second claims for the second member.

4. The method of claim 1, further comprising:
determining a third member is not valid for reassignment based on third member information indicating that the third member is enrolled into a first program and a second program.

5. The method of claim 1, further comprising:
determining a fourth member is not valid for reassignment based on fourth member information indicating that the fourth member is a newborn baby.

6. The method of claim 1, further comprising:
determining a fifth member is not valid for reassignment based on fifth member information indicating that the fifth member is currently assigned to a primary care provider (PCP) that has been terminated.

7. The method of claim 1, wherein processing the first member information to confirm that the first member is assigned to the first medical provider comprises processing the first member information to confirm that the first member is actively enrolled or has a future enrollment with a primary care provider (PCP).

8. The method of claim 1, further comprising:
determining a plurality of valid providers based on one or more provider criteria, and
wherein obtaining the one or more claims for the first member is based on determining the plurality of valid providers.

9. The method of claim 8, wherein the one or more provider criteria indicates one or more panel thresholds, and wherein determining the plurality of valid providers comprises:
comparing panel sizes indicating a number of members that are currently under care by the providers with the one or more panel thresholds; and
determining the plurality of valid providers based on the comparison.

10. The method of claim 1, wherein obtaining the one or more claims for the first member within the first time period comprises:
retrieving, from a claims systems, claims data comprising a plurality of claims associated with the first member;
filtering the claims data to determine the one or more claims for the first member based on comparing dates of service for the plurality of claims with a fourth time window; and
storing the one or more claims for the first member within a claims database, wherein reassigning the first member to the second medical provider is based on the one or more stored claims within the claims database.

11. The method of claim 1, wherein the one or more claims comprises a plurality of claims for the first member, and wherein reassigning the first member from the first medical provider to the second medical provider comprises:
determining a claim, from the plurality of claims, that has a date of service that is most recent to a current run-time date, wherein the determined claim indicates the second medical provider; and
reassigning the first member from the first medical provider to the second medical provider based on the determined claim indicating the second medical provider.

12. The method of claim 1, wherein the one or more claims comprises a plurality of claims for the first member, and wherein reassigning the first member from the first medical provider to the second medical provider comprises:
determining a plurality of addresses associated with a plurality of providers that are indicated by the plurality of claims for the first member;
based on comparing the plurality of addresses associated with the plurality of providers to an address associated with the first member, determining the second medical provider that has a closest geographical distance to the address associated with the first member; and
reassigning the first member from the first medical provider to the second medical provider based on determining that the second medical provider the closest geographical distance to the address associated with the first member.

13. The method of claim 1, wherein the one or more claims comprises a plurality of claims for the first member, and wherein reassigning the first member from the first medical provider to the second medical provider comprises:
determining a plurality of panel sizes associated with a plurality of providers that are indicated by the plurality of claims for the first member;
based on a comparison of the plurality of panel sizes, determining the second medical provider that has a smallest panel size from the plurality of panel sizes; and
reassigning the first member from the first medical provider to the second medical provider based on determining that the second medical provider has the smallest panel size from the plurality of panel sizes.

14. The method of claim 1, wherein informing the first member of the reassignment from the first medical provider to the second medical provider comprises performing a process to mail a new insurance card to the first member that indicates the second medical provider.

15. A computing platform for reassigning a plurality of members from one or more currently assigned medical providers to one or more newly assigned medical providers, comprising:
one or more processors; and
a non-transitory computer-readable medium having processor-executable instructions stored thereon, wherein the processor-executable instructions, when executed by the one or more processors, facilitate:
identifying a first member for reassignment based on determining that the first member is valid for reassignment, wherein determining that the first member is valid for reassignment comprises:
processing first member information to confirm that the first member is assigned to a first medical provider;
determining that the first member has not been identified for reassignment by the computing platform within a first time window; and
determining that an earliest effective enrollment date for the first member is outside of a second time window;
obtaining, from a claims repository, one or more claims for the first member within a first time period, wherein the one or more claims are medical claims associated with a primary care provider (PCP);
based on the one or more claims indicating a second medical provider that is different from the first medical provider, reassigning the first member from the first medical provider to the second medical provider; and
informing the first member of the reassignment from the first medical provider to the second medical provider.

16. The computing platform of claim 15, wherein determining that the first member is valid for reassignment further comprises:
determining that the first member has not been previously reassigned by the computing platform, and wherein determining that the earliest effective enrollment date for the first member is outside of the second time window is in response to determining that the first member has not been previously reassigned by the computing platform.

17. The computing platform of claim 15, wherein the processor-executable instructions, when executed by the one or more processors, further facilitate:
identifying a second member for reassignment based on determining that the second member is valid for reassignment, wherein determining that the second member is valid for reassignment comprises:
processing second member information to confirm that the second member is assigned to a third medical provider;
determining that the second member has not been identified for reassignment by the computing platform within the first time window;
determining that the second member has been previously reassigned by the computing platform; and
in response to determining that the second member has been previously reassigned, comparing a reassignment date for when the member was previously reassigned with a third time window; and
based on determining that the second member is valid for reassignment, reassigning the second member from the third medical provider to a fourth medical provider associated with one or more second claims for the second member.

18. The computing platform of claim 15, wherein the processor-executable instructions, when executed by the one or more processors, further facilitate:
determining a third member is not valid for reassignment based on third member information indicating that the third member is enrolled into a first program and a second program.

19. The computing platform of claim 15, wherein the processor-executable instructions, when executed by the one or more processors, further facilitate:
determining a fourth member is not valid for reassignment based on fourth member information indicating that the fourth member is a newborn baby.

20. A non-transitory computer-readable medium having processor-executable instructions stored thereon, wherein the processor-executable instructions, when executed, facilitate:
identifying a first member for reassignment based on determining that the first member is valid for reassignment, wherein determining that the first member is valid for reassignment comprises:
processing first member information to confirm that the first member is assigned to a first medical provider;
determining that the first member has not been identified for reassignment by a claims-based reassignment computing platform within a first time window; and
determining that an earliest effective enrollment date for the first member is outside of a second time window;
obtaining, from a claims repository, one or more claims for the first member within a first time period, wherein the one or more claims are medical claims associated with a primary care provider (PCP);
based on the one or more claims indicating a second medical provider that is different from the first medical provider, reassigning the first member from the first medical provider to the second medical provider; and
informing the first member of the reassignment from the first medical provider to the second medical provider.
